(19) United States
(12) Patent Application Publication
(10) Pub. No.: US 2003/0017600 A1
(43) Pub. Date: Jan. 23, 2003

(54) Double selection vector

(76) Inventors: Philippe Gabant, Brussels (BE); Claude Szpirer, Waterloo (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/168,774

(22) PCT Filed: Dec. 20, 2000
(86) PCT No.: PCT/BE00/00151

(30) Foreign Application Priority Data
Dec. 20, 1999 (EP) 998702682

(51) Int. Cl. C12N 15/74; C12N 1/21

(52) U.S. Cl. 435/472; 435/320.1; 435/252.3

(57) ABSTRACT

The present invention is related to a nucleic acid construct (1) to be incorporated in a double selection vector (2) able to transform a cell (3) of a specific organism, wherein—said construct (1) contains two different genes (10 and 11), each gene encoding a different toxic molecule (4 and 5) to a prokaryote cell, preferably to E. Coli, said genes (10 and 11) being disposed upstream and downstream a cassette sequence (8), or downstream and upstream site(s) for the insertion of a cassette sequence (8), and—said nucleic acid construct comprises specific sequence portions (12, 12′) allowing inactivation of said genes (10 and 11).

### FIELD OF THE INVENTION

The present invention is related to a nucleic acid construct and a double selection vector, its host cell, its preparation process and its use for the modification, the deletion or the replacement of genes in order to obtain non-human genetically modified unicellular or pluricellular organisms, preferably knock-in or knockout organisms such as a knock-in or knock-out mouse.

### BACKGROUND OF THE INVENTION

Plasmid cloning vectors are commonly used for the propagation and amplification of DNA fragments in Escherichia coli. The insert of interest is ligated with the plasmidic vector linearized by a restriction endonuclease generating compatible ends. After transformation with ligation mix, experimentators face the problem of separating transformants that acquired a recombined plasmid from bacteria that contain the parental vector. To minimize the number of transformants due to self-ligated plasmid molecules, positive selection vectors were developed (for example, see Vernet et al., 1985; Kuhn et al., 1986; Gossen et al., 1992; Guilfoyle and Smith, 1994; Henrich and Schmidtberger, 1995; Trudel et al., 1996). Efficient positive selection cloning vectors were developed using CcdB (control of cell death B), a gene of the F plasmid coding for a natural poison acting on the E. coli gyrase (Bernard et al., 1994 and U.S. Pat. No. 5,910,438. The ccd operon belongs to the poison-antidote systems found in different natural large plasmids (Jensen and Gerdes, 1995; Couturier et al., 1998). These poison-antidote loci (also termed post-segregational killing systems) contribute to the stability of low copy number plasmids in bacterial populations by killing daughter cells that have not inherited a plasmidic copy.

The main advantages of the ccdB-containing vectors over the other positive selection systems are i) the small size of their selective gene (ccdB: 303 bp), ii) the fact that the vector can be amplified in a host harboring a mutation that confers total resistance to the CcdB poison (gyrA462 resistant strain ; Bernard and Couturier, 1992). Since E. coli is the host used for most molecular cloning strategies, it is important to develop new systems which can enrich and widen the range of cloning possibilities. The positive selection technology using CcdB has been used to derive new vectors adapted to peculiar purposes: PCR cloning vectors (Gabant et al., 1997), vectors adapted for bacterial genetics (Gabant et al., 1998), and recently a kid gene belonging to the CcdB family has been used to design new cloning vectors (Gabant et al., 2000).

In the field of genomics, a lot of new genes and complete genomes are cloned and sequenced. In order to assess the biological functions of these new coding sequences, their corresponding genes must be specifically mutated (deleted or modified) in the genome of an organism (for instance in a knock-out mouse), which allows the study f phenotype(s) related to the mutation introduced. The specificity of the mutation is given by a targeting vector (constructed in E. coli) containing homologous recombination arms.

However, the construction in E. coli of a vector containing recombination arms for homologous recombination before transfection into pluripotent cells or somatic cells can present several drawbacks. It is a long and complicated proceeding that requires several cloning steps for the insertion of said homologous recombination arms on each side of the cassette nucleotide sequence to be inserted in said pluripotent or said somatic cell.

### AIMS OF THE INVENTION

The present invention aims to provide a new nucleic acid construct and a hereafter called “double selection vector” as well as its host cell, suitable for new applications in the field of post-genomics research.

A particular aim of the present invention is to provide a nucleic acid construct and a vector suitable for the incorporation of recombination arms in correct orientation therein and which could be used thereafter in homologous recombinations allowing a modification, a replacement or a deletion of a target nucleotide sequence from the genome of a totipotent or a somatic cell.

Another aim of the present invention is to provide the corresponding cell wherein said target nucleotide sequence has been modified, replaced or deleted and possibly the corresponding organism made of or having incorporated said cell(s) and comprising in its genome the modification, the replacement or the deletion of said specific target nucleotide sequence.

A last aim of the invention is to provide a simple and possibly automatic method of conception and/or production of said vector adapted for all types of modification, replacement or deletion of a specific target nucleotide sequence from the genome of a cell.

### SUMMARY OF THE INVENTION

The present invention is related to a vector 2 able to transform a cell 3 (which means a vector comprising all the elements suitable for the transformation of a cell with its incorporated genetic sequence) and comprising at least two different genes (10 and 11), each encoding different toxic molecules (4 and 5) to a prokaryote cell, preferably to E. coli.

Said genes (10 and 11) are preferably disposed in the vector 2 in opposite and confluent lecture orientations to each other.

The present invention is also related to a nucleic acid construct 1 to be incorporated in said vector 2 able to transform a cell 3 of a specific organism; said construct 1 containing two different genes (10 and 11), (each encoding a different toxic molecule (4 or 5) to a prokaryote cell, preferably to E. coli, said genes (10 and 11) being disposed upstream and downstream a cassette sequence 8 or upstream and downstream of one or more site(s) allowing the insertion of said cassette 8 (i.e. restriction site or site for recombinase).

Preferably, said genes are disposed in opposite and confluent lecture orientations to each other (upstream and downstream said cassette sequence 8), as illustrated in the enclosed FIGS. 3 and 4.

Said nucleic acid construct comprises also specific sequence portions (12 and 12′) allowing the inactivation of said genes, preferably through insertion of a foreigner sequence in said genes or through a partial or total deletion of said genes, preferably by homologous recombination or by the use of recombinases.

According to a first preferred embodiment of the present invention, the cassette sequence 8 may be integrated into the nucleic acid construct 1 or vector 2 through a fusion by a clonase of two separate vectors (see FIG. 4).

According to a second embodiment of the present invention, the specific sequence portions (12 and 12′) of the nucleic acid construct allowing the inactivation of genes (10 and 11) are sequences which comprise several or unique cloning sites present in said genes and/or in their operator/promoter sequence, allowing the cleavage by restriction enzymes and allowing the insertion inside said genes (10 and 11) of one or more foreigner sequences, such as the recombination arms (15 and 16), as described hereafter (see FIG. 3).

According to a third preferred embodiment of the present invention, the specific sequence portions could be sequences including specific sites recognised by recombinase (i.e. sites disposed upstream and downstream the genes or the portions of the genes to be deleted and recognised by said specific recombinase), and are for instance “att phage λ base sites” (specific recombination such as the one described by Ptashne.M (Genetic Switch, Cell Press, Cambridge 1992)). Preferably, said “att sites” are integrated in the nucleic acid construct according to the invention according to the method described by Landy A. (Annual Review Biochemistry, Vol. 58, p. 913 (1989)). Preferably, said att sites integrated in the nucleic acid construct comprise 25 base pairs and have the following genetic sequence SEQ ID N°1: ACA ACT TTG TAC AAA AAA GCA GGC T as well as its complementary strand or the sequence SEQ ID N°2: ACC CAG CTT TCT TGT ACA AAG TGG T and its complementary strand.

It is also possible to obtain a nucleic acid construct which may comprise additional different specific sequence portions which can be inactivated by one or more of the above-identified methods (inactivation through an insertion in the genes or through a partial or total deletion of the genes).

“A gene 10 or 11 encoding a toxic molecule 4 or 5 for a prokaryote cell” means a nucleotide sequence comprising a prokaryote (preferably E. Coli) promoter/ operator sequence 9 or 9′ and a sequence encoding said toxic molecule 4 or 5 for said prokaryote cell, preferably for E. Coli.

Each different gene comprised in said nucleic acid construct contains said specific sequence portions allowing the gene inactivation either in the prokaryote promoter/operator sequence 9 or 9′ or into the sequence encoding the toxic molecule 4 or 5 for said prokaryote cell. Said specific sequence portions present in each gene upstream or downstream said genes are different for each gene.

“Two different genes 10 and 11 encoding two different toxic molecules 4 and 5” means that said two toxic molecules are able to reduce the growth or are able to kill said prokaryote cell and are independent from each other (i.e. their modes of action are obtained by two different biological mechanisms, even if those mechanisms kill by acting on the same gene product), which results in the killing of said prokaryote cell or affects its growth.

“A prokaryote promoter/operator sequence 9 or 9′” is a regulatory sequence obtained from a prokaryote cell, preferably from E. Coli, and comprising preferably the nucleotide sequence described in FIG. 1 for sigma 70 E. coli promoter.

According to the invention, the two toxic molecules 4 and 5 for a prokaryote cell, are advantageously two poison proteins (encoded by a wild type or modified nucleotide sequence) which are naturally or artificially poisonous and affect one or more vital functions of a prokaryote cell, preferably E. Coli.

A protein poison is also characterised by the existence of an antidote or an anti-poison such as the protein CcdB and its antagonist CcdA, the protein Kid and its antagonist Kis, the protein Doc and its antagonist Phd, the protein HoK and its antagonist SoK, relE toxin and its anti-toxin RelB, PasA and its antidote PasB and PasC, mazE and its antidote mazF, and other poison molecules which are or are not of plasmid origin.

Other examples of toxic molecules for a prokaryote cell are the protein encoded by the gene sacB (from Bacillus amylolique-faciens), the protein GpE, the protein GATA-1 or the protein Crp. The gene sacB encodes the levan sucrase which catalyzes the hydrolysis of sucrose into products which are toxic for E. Coli (Pierce et al. Proc. Natl. Acad. Sci., Vol. 89, N°6 (1992) p. 2056-2060). The protein GpE encodes the E genes from the bacteriophage φX174 which includes six unique restriction sites and encodes gpE and which causes lysis of E. Coli cell (Heinrich et al., Gene, Vol. 42 n°3 (1986) p. 345-349). The protein GATA-1 has been described by Trudel et al. (Biotechniques 1996, Vol. 20(4), p. 684-693). the protein Crp has been described by Schlieper et al. (Anal. Biochem. 1998, Vol. 257(2), p. 203-209).

“A cassette sequence 8” is a sequence of DNA containing one or more selectable genes expressed in the eukaryote cell. the insertion of such sequence into the genome of the cell confers to this organism a selectable property (for example the resistance to an antibiotic like Neo giving a resistance to Geneticin). Said cassette may further contain sequences to be expressed in the host cell, allowing the detection of cells by reporter genes (such as LacZ, GFP, etc.) or specific recombinases (such as Cre of P1). This cassette may also contain site allowing total or partial deletion(s) of DNA by action of recombinases (such as LoxP sites for Cre). Finally, this cassette may also consist or contain any coding sequence including the original sequence comprising one or more specific mutations or deletions to be expressed in the eukaryote cell after homologous recombination.

Alternatively, said cassette 8 may contain a site (recognition sequence for a restriction enzyme and/or recombinase) allowing the insertion of a selectable gene.

Preferably, the nucleotide sequence incorporated in the two genes 10 and 11 corresponds to the following sequences SEQ ID NO. 1 and SEQ ID NO. 2, comprising respectively two fusion proteins poisons made of a polylinker 12 comprising several unique cloning sites different for each sequence and a nucleotide sequence encoding either the protein poison CcdB or the protein poison Kid.

Another aspect of the present invention is the vector 2 (preferably a viral or plasmid vector such as the pUC18 vector) comprising said nucleic acid construct and all the necessary elements for the replication of said vector in a prokaryote cell, preferably in E. Coli.

A further aspect of the present invention is related to the prokaryote cell transformed by said vector, preferably a prokaryote cell “which is resistant to one of the two toxic molecules 4 and 5 expressed by said vector”. Preferably, said prokaryote cell (IBMM140) comprises a gene encoding an antidote to one of said toxic (poison) molecules, preferably a gene encoding the molecule Kis which is the antidote to the protein Kid or to a fusion protein comprising said protein Kid.

The present invention is also related to the host cell of said nucleic acid construct and said double selection vector, preferably a prokaryote cell, which is resistant to the two toxic molecules (4 and 5) encoded by the two genes (10 and 11). Preferably, said prokaryote cell possesses mutation(s) that confers a resistance to the toxic activity of said first and/or said second toxic molecule(s) and/or possesses one or more genes that encode one or more antidote(s) to said first and/or said second toxic molecule(s); with the provisio that said cell does not possess only a mutation which confers resistance to the toxic activity of the poison CcdB molecule only (or corresponds to the strain LMGP-12601 described in the U.S. Pat. No. 5,910,438).

Advantageously, said prokaryote host cell (IBMM150) having the deposit number LMGP-19171 (filed on Nov. 29, 1999) possesses a mutation wherein the Arginine 462 is replaced by a Cysteine in the amino acid sequence of the GyrA polypeptide of the gyrase (see U.S. Pat. No. 5,910,438) and possesses the genetic sequence encoding the protein Kis that is an anti-poison of the protein Kid.

Another aspect of the present invention is related to a host cell of said nucleic acid construct but which is resistant to one toxic molecule 5 encoding by one gene 11 or a process gene that encoded an antidote to said toxic molecule 5 with the provisio that said toxic molecule is not CcdB. Preferably, said cell comprises a gene that encoded the antidote Kis to the toxic molecule Kid.

According to another embodiment of the invention, the double selection vector according to the invention further comprises, inserted in one of the cloning sites of the first gene 4, a first recombination arm 15 and at one of the cloning sites of the second gene 5, a second recombination arm 16.

Said double selection vector 2 having incorporated said recombination arms (15 and 16), hereafter called “targetor vector”, allows the insertion of a portion (comprising the cassette sequence 8) of the nucleic acid construct 1 according to the invention in the genome of a transformed cell 3. The “targetor vector” having integrated the recombination arms (15 and 16) in a correct orientation in the nucleic acid construct 1 according to the invention (after its cloning in E. coli) is selected and recovered. Thereafter, said “targetor vector” is used to obtain an homologous recombination between the two recombination arms (15 and 16) and corresponding nucleotide sequences present in the genome of the cell, said sequences being disposed upstream and downstream the target sequence to be modified, deleted or replaced (as illustrated in FIG. 2).

Therefore, a further aspect of the present invention is related to the cell 3 (totipotent cell or somatic cell), preferably a pluripotent embryonic stem (ES) mice cell, in which a target nucleotide sequence has been modified, deleted or replaced by the portion (comprising the cassette sequence 8) of the “targetor vector” according to the invention.

Another aspect of the present invention is related to the corresponding non-human pluricellular organism (selected from the group consisting of micro-organisms, fungi (including yeast), plants or non-human animals (preferably a mammal such as a mouse) made of said cells or having incorporated said cells (for instance in a chimera) and comprising in its genome the above-described deletion or modification of a target nucleotide sequence. Said cell and non-human organism can be identified by a genetic analysis of the incorporated foreigner genetic fragments (i.e. fragments of the nucleic acid construct 1 according to the invention), especially one or more prokaryote promoter/operator sequences (9 and 9′), preferably obtained from E. coli, in particular prokaryote promoter/operator sequences (9 and 9′) disposed in opposite divergent lecture orientation upstream and downstream a cassette sequence 8.

A further aspect of the present invention is related to a (possibly automated) method for the replacement/the modification of the deletion of a target genetic sequence into an eukaryote cell 3 by a cassette sequence 8, comprising the steps (possibly performed by an automate) of:

possibly selecting said target genetic sequence from a genome databases (GDB:http://gdbwww.gdb.org, by using algorithms of OMIM™, FGENE™, geneFinder™, HMMGene™, Genscan™) through analysis of said genomic sequence by the identification of exon-intron structure in said genomic sequence and comparison with expression genetic databases (including ESTs) for instance presented on EMBL Genbank and Swiss prot™ databases, bibliographic databases including patent databases, by using algorithms of Blast™, Fasta™, Repeatmasker™, possibly based upon specific requests, such as, the compatibility with another vector or shuttle vector, the stability, the food safety, specific applications such as cloning, expression or targeting, etc. sent by Internet by the customer.

possibly providing the suitable configuration of the vector and the primer sequences suitable for the amplification and cloning of said target genetic sequences (for instance by using the WWW 2GCG™ database),

possibly providing and selecting the design of a vector or a nucleic acid construct according to the invention, and comprising the nucleotide sequence 8 disposed between suitable recombination arms (15 and 16),

possibly recovering the design of the obtained virtual vector or nucleic acid construct into a target memory database,

possibly obtaining the suitable means (selected primers, buffer media, DNTPS, PCR cyclers™, electrophoresis media and devices, means and media for the selection of the recombinant bacteria, specific host cells, etc.) for the preparation of the selected nucleic acid and the vector according to the invention (see FIG. 5),

incorporating the nucleic acid construct 1 according to the invention into a suitable vector 2 (suitable for the transfection of prokaryotic cells),

possibly submitting said vector (2) (if it is circular) to a cleaving action (preferably by the action of a restriction enzyme, recombinase or any other means) upon the first specific sequence portions, preferably by the action of a restriction enzyme upon the “unique cloning” site 12 of the nucleic acid construct 1 incorporated in said vector), allowing the cleavage or the partial deletion of the first gene 10,

incorporating into said vector 2 a first recombination arms 15 that desactivates the toxic activity of the nucleotide sequence present in the first gene 10,

selecting the vector having integrated in the correct orientation the first recombination arm 15 (by transforming a strain (IBMM140) which is sensible to the toxic activity of the first gene 10, but which is resistant to the toxic activity of the second gene 11), and control by PCR electrophoresis checking,

submitting the recovered vector to a cleaving action upon the second specific sequence portions (preferably to the action of a restriction enzyme upon the unique cloning site 12′ of the nucleic acid construct 1 incorporated in said vector), allowing the cleavage or the partial or total deletion of the second gene 11,

incorporating into said vector 2 a second recombination arm or a vector comprising it, that desactivates the toxic activity of the nucleotide sequence present in the second gene 11,

selecting the vector (possibly after the use of a clonase) having integrated in the correct orientation the second recombination arm 16 (by transforming a strain that is sensible to the toxic activity of the second gene 11), and control by PCR electrophoresis checking,

cloning said vector 2 in a prokaryote cell, preferably in E. coli (see FIGS. 3 to 7), and

transforming a totipotent or a somatic cell 3 in conditions allowing the replacement of a target nucleotide sequence present in the genome of said cell by the cassette sequence 8 of the nucleic acid construct 1 present in said vector 2 by homologous recombination between the arms (15 and 16) and corresponding nucleotide sequences present upstream and downstream the target sequence to be modified or deleted in the genome of said cell 3,

recovering the somatic or totipotent cell 3 wherein the target sequence has been replaced by the cassette sequence 8, and

possibly obtaining a pluricellular organism made of said cell 3 or having incorporated said cell 3 and comprising in its genome the deletion or the modification of the target nucleotide sequence, replaced by the cassette sequence 8 of the nucleic acid construct 1 according to the invention.

Preferably, one or more of the above-identified steps, especially the ones for the selection of the target genetic sequences from databases, the design and providing of the vector and primer and the recovering of the design of sail virtual vector could apply to all types of vector configuration and not only the double selection vector according to the invention (see FIG. 5).

The present invention is also related to a kit of parts or a device comprising one or more of the above-described products as media and means suitable for performing the method according to the invention, preferably the nucleic acid construct 1 according to the invention, advantageously incorporated in the vector 2 according to the invention. Said vector is preferably linear and has been cleaved by a restriction enzyme upon one of the unique cloning sites 12 of the nucleic acid construct 1 incorporated in said vector 2. The kit according to the invention may comprise also a prokaryote cell for said vector, preferably a prokaryote host cell, that is resistant to the toxic activity of the second toxic molecule 5 or possesses a gene that encodes one or more antidotes to said second toxic molecule 5, said second toxic molecule 5 being encoded by the gene 11 that has not been cleaved by the restriction enzyme above-described, with the provisio that said cell does not comprise only a resistance to toxic activity of the toxic molecule CcdB or a gene that encode the antidote CcdA to said toxic molecule CcdB (preferably said strain corresponds to the strain MGP-12505 described in the U.S. Pat. No. 5,910,438 incorporated herein by reference). The above means could comprise also computer programmes for performing one or more of the various steps of the method according to the invention (including the identification of the target genetic sequence from genome databases and the design of the nucleic acid construct, its primers and the vector).

Therefore, a last aspect of the present invention is also related to a computer program comprising program code means for performing these steps or all the steps of the method according to the invention above described, when said program is run on a computer.

The present invention is also related to a computer program product comprising program code means stored on a computer readable medium for performing one or more or all the steps of the method according to the invention above described, when said program is run on computer.

Said program could comprise also specific means for presenting one of the steps or all the steps to be performed for obtaining the nucleic acid construct and its vector as well as the design upon a specific interface with the consumer, preferably upon a web site or upon a PC linked to an automate performing one or more of the various steps according to the invention.

In the device and method according to the invention, the prokaryote cell is the strain IBMM140 that is resistant to the Kid protein and is competent (able to receive DNA under the form of the vector according to the invention). This competence can be obtained by a chemical treatment or by a proceeding allowing electroporation of the strain(such as the one described by Ausubel et al. (Current Protocols in Molecular Biology, New York, Greene Publishing Associates and Whiley-Interscience (1994)).

Furthermore, said sensibility to the toxic activity of the second toxic molecule 5 encoded by the gene 11 is advantageously inducible by a metabolite. For instance the strain IBMM140 allows advantageously the production of the antidote Kis in the presence of an increased concentration of arabinose.

The kit according to the invention may further comprise culture medium (under solid or liquid form) and other means for improving the specific method according to the invention (for instance sequencing and/or amplification primers, restriction or recombinase (clonase™ mix from Gibco-BRL) enzymes, DNA polymerase, DNA ligase, etc.) as well as other specific saline solution buffers containing the nucleotides, control plasmids and specific eukaryote cells to be transformed (for instance ES cells).

### SHORT DESCRIPTION OF THE DRAWINGS

FIG. 1 represents a promoter consensus sequence defined for a sigma 70 E. coli promoter.

FIG. 2 represents the nucleic acid construct according to the invention integrated into a plasmid.

FIGS. 3 to 8 represent schematically several steps used in the method according to the invention for the replacement of a target genetic sequence into a cell by a cassette sequence.

### DETAILED DESCRIPTION OF THE INVENTION

Description of Plasmids and Strains

The nucleic acid construct 1 comprises, preferably integrated into a plasmid 2 (for instance of the type pUC18), two positive selection sequences (corresponding to the above-described genes 4 and 5) disposed preferably in opposite divergent lecture orientation upstream and downstream a cassette sequence 8 The first positive selection sequence 4 incorporating the CcdB sequence has been described by Bernard P. et al. (Gene 148, pp. 71-74 (1994)). The other positive selection sequence 5 is based upon the use of the parD killer gene encoding the kid poison toxin.

The kis coding sequence was amplified by PCR on the plasmid R1 drd19 (Meynell and Datta, 1966), using the following primers: kis1 -5′gaggaattctggaggtgaagaatgcatac3′- and kis2 -5′gagaagctttcagatttcctcctgaccag3′-. The resulting product was cloned in TOPO activated pCR-XL™ vector (Invitrogen, Carlsbad, Calif., USA), and this insert was then subcloned in pBAD33 (Guzman et al., 1995) to give pBAD-kis. To construct λ::kis, the kis gene, placed under the control of Pbad promoter was amplified by PCR from pBAD-kis using the following primers: λ1 -5′tagagatctgatgcataatgtgcctgtc3′- and λ2: -5′tagagatctgagcaaaaacaggaaggc3′-. The resulting product was cloned in TOPO activated pCR-XL. This insert was then cloned in pRS551 (Simons, et al., 1987) open by BamHI to generate pRS-kis. The MM294 (Backman and Boyer, 1983) strain containing pRS-kis was infected by XRS45 and recombinants were selected on LB-kanamicyn as described by Simons, et al., (1987) in LVM103 F′ traD36 proAB lacI^{q} lacZΔM15/Δara leu::Tn10 supE thi-1 Δ(lac-proAB). This strain, lysogenized by λ::kis was called IBMM120. To construct Tn::kis, the kis gene placed under the control of the Pbad promoter and the chloramphenicol resistance gene were amplified by PCR from pBAD-kis using the following primers: Tn1 -5′taggcggccgcgatgcataatgtgcctgtc3′- and Tn2: -5′taggcggccgccagaagccactggagcacc3′-. The resulting product was cloned in TOPO activated PCR-XL and subcloned in the unique SfiI site of the suicide vector pMF100, a Δ Km derivative of pUT/Km (Herrero et al., 1990) The pUT-kis plasmid was transformed in S17-1 (λ-pir): pro82 rfbD1 spoT1 supE44 endA1 hsdR17 recA [RP4-2-Tc::Mu-Km::Tn7] (λ-pir) (de Lorenzo via Van der Lelie). This strain was mated with IBMM139 (LVM103 λ resistant) and a clone, IBMM140, with a Tn::kis transposed in the chromosome, was isolated.

Construction of the Vectors pKID18 and pKID19

parD is a poison-antidote stabilization system of the R1 plasmid, a member of the IncFII plasmid family. This locus participates in the maintenance of R1 by postsegregational killing of plasmid-free bacteria and consists in a small operon containing two genes: kid (333 bp) and kis (258 bp) coding for a killer component (Kid) and its antagonist (Kis), respectively (Bravo et al., 1988). This system is perfectly conserved and functional in another IncFII plasmid, R100 (pem system: Tsuchimoto et al., 1988), which contains the genes termed pemI (identical to kis) and pemK (identical to kid). The structure and function of parD are similar to those described for ccd of the F plasmid. Using the R1 kid killer gene, we constructed new cloning vectors allowing positive selection of recombinants. The kid sequence was fused in frame with the multiple cloning site (MCS) of either pUC18 or pUC19 (Yanish-Perron et al., 1985) to generate pKID18 and pKID19 respectively.

Both pKID18 and pKID19 express a fused Kid protein under the control of the lactose promoter (Plac). These plasmids were amplified in TOP10F′ bacteria [F′ lacI^{q} Tc ^{R}/mcrA Δ(mrr-hsdRMS-mcrBC) φ80 lacZΔ M15 ΔlacX74 deoR RecA1 araD139 Δ(ara-leu) 7697 galU galK rpsL endA1 nupG] (Invitrogen, Carlsbad, Calif., USA) In this strain, the kid fusions are transcriptionally repressed by the host lacI^{q} repressor.

In order to avoid any selective advantage for inactive mutants in the kid selective marker during amplification, it is essential to amplify these vectors in a host insensitive to Kid poisoning. Bacterial strains expressing Kis are host for kid-based vector amplification.

Two strategies may be used: the kis gene was inserted into a lambda vector (λ::kis) or into a one hooper transposon (Tn::kis) carried by a suicide vector. Strains lysogenic for λ::kis or containing a copy of Tn::kis in the chromosome were isolated. The two resulting strains IBMM120 and IBMM140 were shown to be completely resistant to pKID18/19 poison activity upon induction of the chromosomal kis gene.

pKID Vectors and Positive Selection

The insertion of a DNA fragment into the multiple cloning site of pKID18/19 leads to disruption of kid genetic information and thus to inactivation of the toxin. Consequently, only bacteria harboring recombinant plasmids should give rise to viable colonies, whereas those bearing the parental vectors should be killed.

CcdB and Kid Selections are Independent

In order to determine whether CcdB and Kid poisoning are independent, one may determine whether the B462 strain coding for a CcdB insensitive gyrase and therefore resistant to CcdB killing, is killed by fused Kid proteins and vice-versa, whether the IBMM120 and IBMM140 strains resistant to Kid upon induction of kis, are killed by fused CcdB proteins. Results show that the CcdB insensitive gyrase (gyrA462) does not protect against Kid poisoning and synthesis of Kis (IBMM120 and IBMM140) does not prevent the killing by CcdB. A double resistant strain was constructed by introducing the gyr462 mutation (CcdBR) (see U.S. Pat. No. 5,910,438) into the IBMM140 strain (Kid^{R}) This new strain called IBMM150 and having the deposit number LMGP-19171 was shown to resist to both systems and thus to constitute the ideal host for the amplification of vectors based either on ccdB, on kid or on both systems.

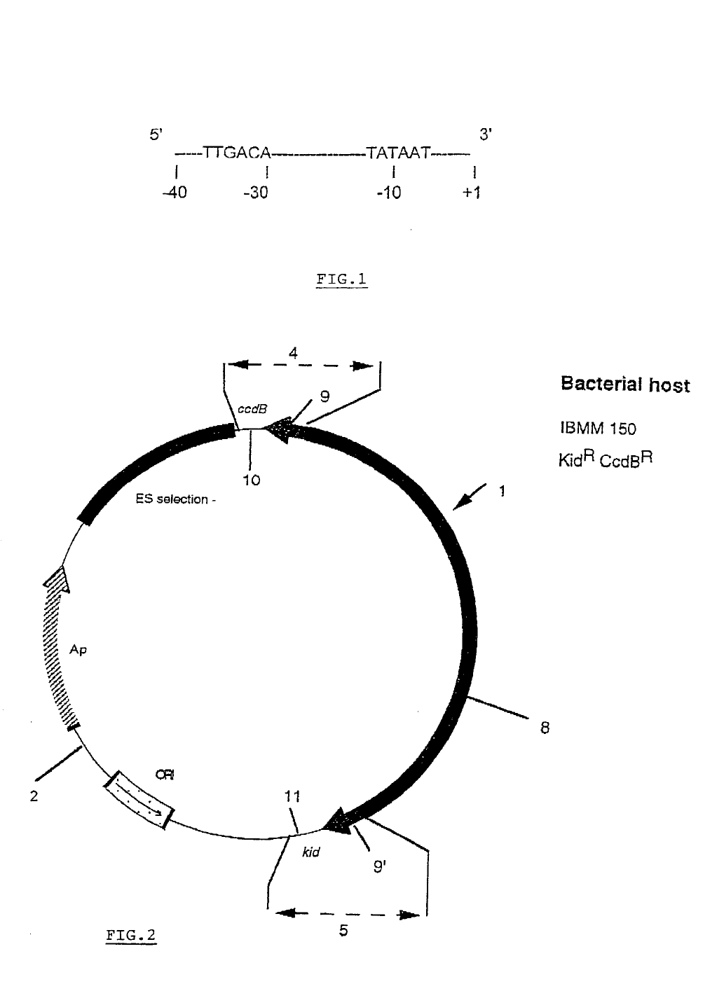

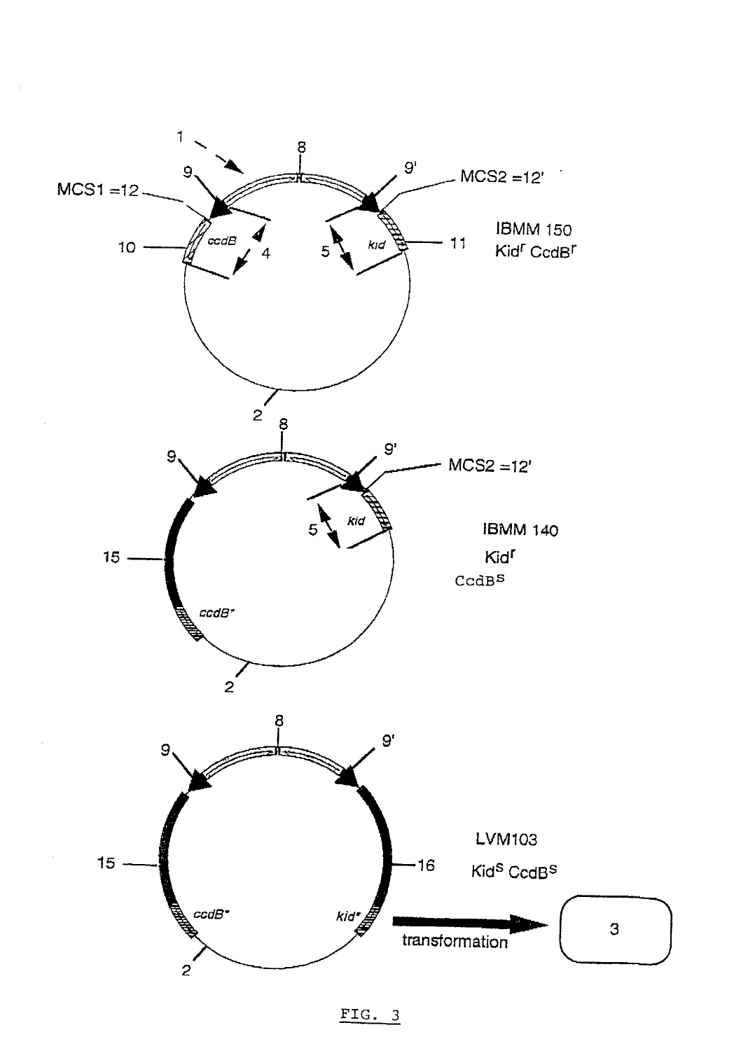

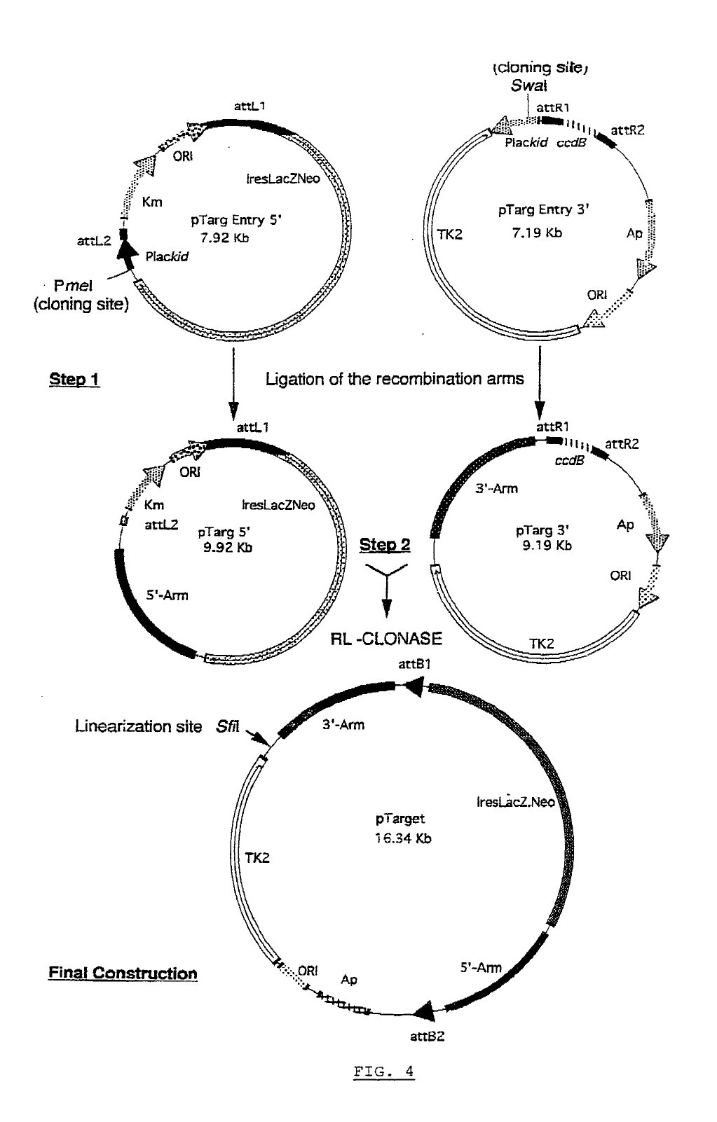

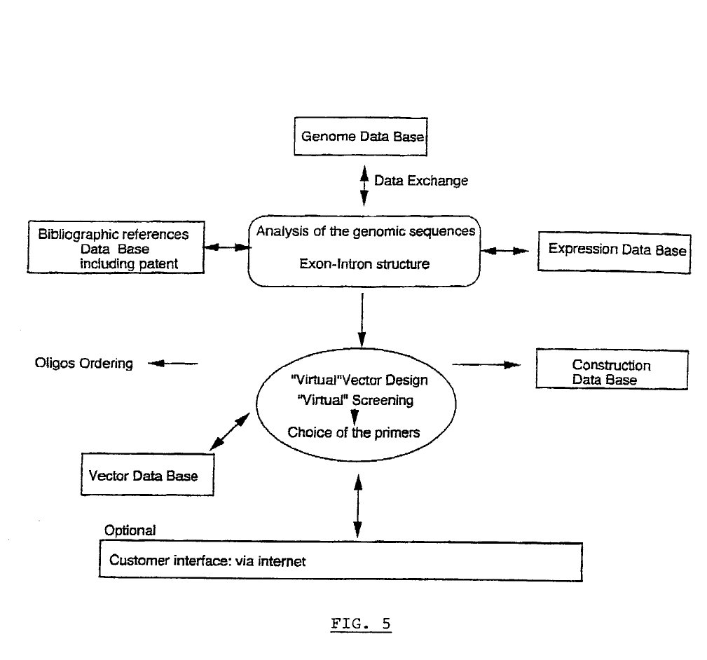

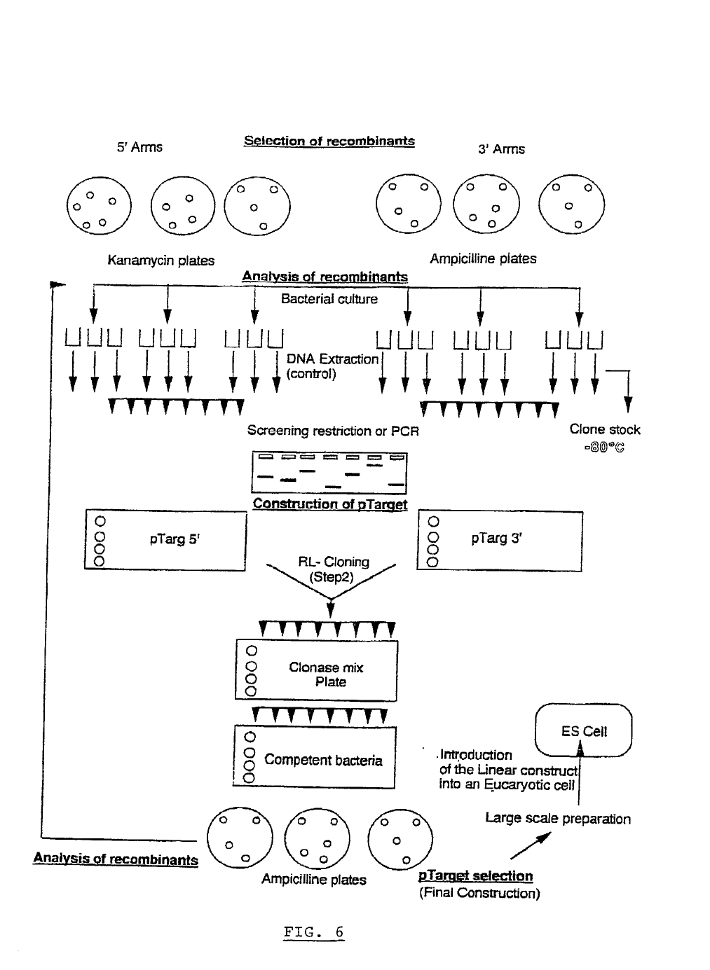

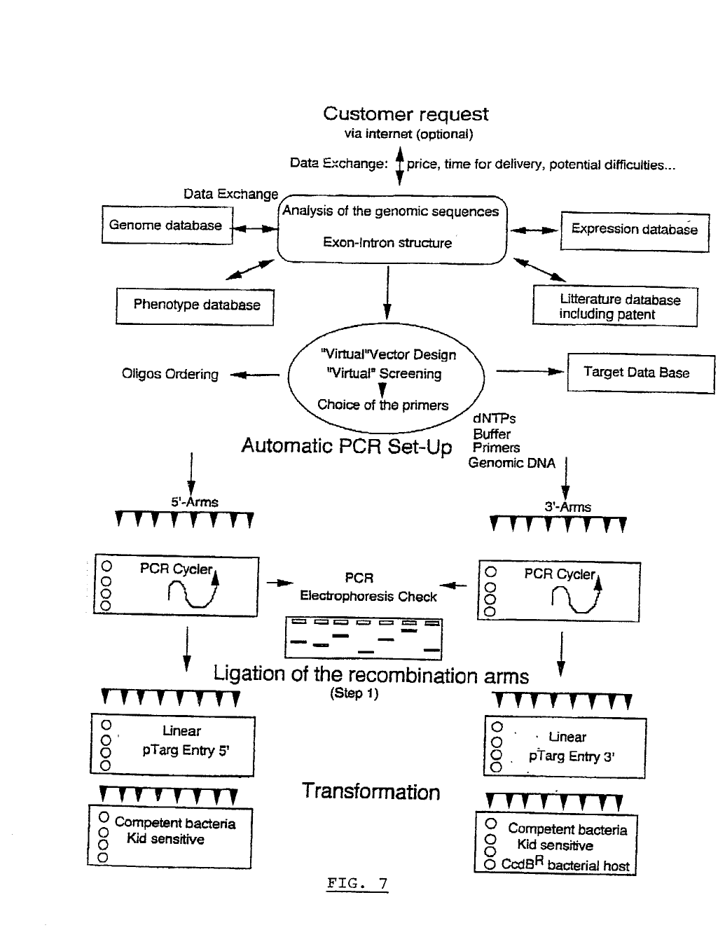

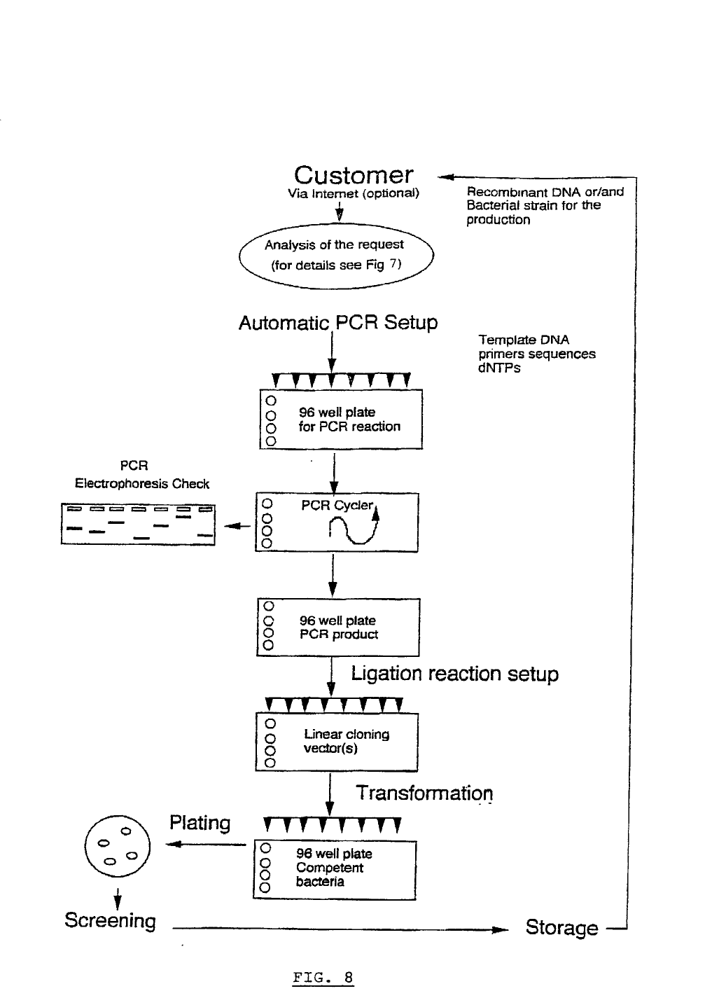

The two above-identified positive selection sequences or genes 4 and 5 according to the invention are incorporated in a plasmid for obtaining the vector 2 as represented in FIG. 1. Said nucleic acid construct is preferably incorporated into a plasmid of the type pUC18 (Yanish-Perron et al., Gene 33, pp. 103-109 (1985)) cleaved by the enzyme Aat2 and AflIII wherein the specific nucleotide sequences 10 and 11 encoding the poison protein CcdB and Kid are introduced after their amplification by PCR. Between said poisonous genes 4 and 5 is cloned a cassette sequence 8 according to the following method. Advantageously, the two multiple cloning sites (MCS) 12 and 12′ in said positive selection sequences (or genes 4 and 5) comprise very rare enzyme restriction sites and comprise also blunt sites that allow insertion of DNA fragments generated by genetic amplification, preferably by PCR. Examples of said enzymes are SwaI, PmeI and SrfI.

In addition, the vector 2 according to the invention comprises also the specific enzyme restriction sites NotI and SfiI integrated between one of the nucleotide sequences 10 and 11 encoding a poison protein and the cassette sequence 8 for a negative selection.

Construction of an Alternative Vector Based Upon the Gateaway TM Technology Construction of pTarg Entry 5′:

i) Construction of the Plac MCS34KID: the multiple cloning site of pKID18 (Gabant et al. 2000) was replaced by the double stranded linker form by the annealing of the following primers: MCS34A: AATTGTTTAAACGCCCGGGCGCGGCCGCGGCGCGCC and MCS34B: AGCTGGCGCGCCGCGGCCGCGCCCGGC-CGTTTAAAC. This linker was cloned into the EcoRI-HindIII of pKID18.

ii) The following sequences were used as primers to PCR amplify the Plac MCS34KID: Plac1: GAGACAGATCTCGCAACGCAATTAATG and KIDAX: GAGACATGTCTCGAGTCAAGTCAGAATAGTGG. The PCR product was cloned into the TOPO activated pCRTOPOXL (Invitrogen).

iii) The pEntry KID34 was obtained by the insertion into the pEntrlA (Life Technologies) open by BamHI and XhoI of the fragment BglII-XhoI containing the Plac MCS 34 KID from the pCRTOPO-XL Plac MCS34KID.

iv) The unique SalI restriction site of pEntry KID34 was replaced by the cloning in this site of the following adapter TCGAAGATCT containing a BglII site.

v) The final pTarg Entry 5′ was obtained by the insertion of a BamHI IRES lacZ Neo selective reporter cassette into the BglII site generated at the step iv.

Construction of pTarg Entry 3′:

i) The NotI-SalI sites flanking the ccdB gene (under its own promoter) was isolated from PENTR 1A (Life Technologies) and cloned into the NotI-SalI sites of pDEST14 (Life Technologies): this construction was named pVAmp.

ii) Construction of PlacMCS12 KID: the multiple cloning site of pKID18 (Gabant et al., 2000) was replaced by the double stranded linker form by the annealing of the following primers: MCS12A: AATTATTTAAATCGCGAGTCGACGGCCC-CAGTGGCC and MCS12B: AGCTGGCCACTGCGGCCGTCGACTCGCGATTTAAAT. This linker was cloned into the EcoRI-HindIII of pKID118.

iii) The following sequences were used as primers to PCR amplify the Plac MCS12KID: Plac1: GAGAGAGATCTCGCAACGCAATTAATG and KIDAX: GAGACATGTCTCGAGTCAAGTCAGAATAGTGG. The PCR product was cloned into the TOPO activated pCRTOPOXL (Invitrogen).

iv) The pVAmp was open by BglII and BspLullI and the PlacMCS12 isolated from the pCRTOPOXL Plac MCS12KID restricted by BglII and EspLullI.

v) The final pTarg Entry 3′ was obtained by the cloning of the XhoI-SalI Tk2 cassette (two copies of the gene coding for the thymidine kinase) into the unique XhoI site of pVAmpKID12.

Properties and Characteristics of the pTarg Entry 3′ Construction

This plasmid contains two selective marker a) Plackid allowing the selection of inserts into the SwaI restriction site and b) a ccdB gene under its own promoter, this last marker is flanked in this plasmid by two sites for the lambda recombinanses (attR1 and attR2). Due to the presence of the two poison genes (ccdB and kid respectively) this plasmid is amplified into a IBMM150 strain (Gabant et al., 2000).

Use of the Targeting Vector According to the Invention in the Preparation of a Knockout Mouse

A clone containing a 129 genomic fragment of AFP loci was isolated from a lambda library. The library was screened with a probe containing the mouse Afp promoter. The genomic insert of about 16 kb was subcloned in the vector 2 according to the invention having incorporated as a cassette sequence the IRES lacZ/neo reporter-selective cassette 8. The obtained vector is also incorporating two recombination arms 15 and 16. The 5′ arms (2.5 kb) was generated by polymerase chain reaction (PCR) using the following primers: N-Mer1: agagcggccgcggaagtgacaaagcagaacc annealing to the MerI sequence of the Afp enhancer 1 (Godboute et al. (1988)) and a primer of the X-exon1: agactcgagggatgagggaagcgggtgtg complementary to the afp exon1. The PCR fragment generated using Pfu polymerase (Stratagene) was cloned in the pCRblunt vector (Invitrogen).

The 3′ arms was subcloned from the lambda into pBSIIKS+ vector (Stratagene) The 5′ recombination arms was introduced upstream the 3′ recombination arms. This construction was electroporated into E14 ES cells 3. Correctly targeted clones were identified by Southern blot analysis using an external probe from the 5′ region.

ES Cell Injections and Animal Genotyping

Recombinant ES cells 3 carrying the targeted allele were injected in C57BL/6J blastocysts. Animals were genotyped by extraction of DNA from tails.

NA Isolation, Northern Blot Analysis

Total RNA was isolated using Trizol (Gibco BRL) extraction according to the manufacturer instructions. For the Northern analysis 20 μg of total RNA were electrophoresed and Transferred to nylon membranes as described. Filters were then hybridized.

Western Blot Analysis

Proteins were separated by SDS-PAGE using 7.5% polyacrylamide gels in a Bio-Rad Mini Protean gel chamber and blotted onto Nitrocellulose filters in a Bio-Rad Trans Blot chamber according to the manufacturer's instructions. Proteins were detected using anti-AFP, anti-Albumin; anti Betagalactosidase serum (ICN Biochemicals) the signal was detected with ECL detection system (Amersham).

LacZ Reporter Gene Expression

To isolate embryonic stages, natural matings were set up and presence of a vaginal plug at noon the following day was taken as 0.5 days of gestation. Staged embryos were stained with X-Gal as wholemounts as described by Forrester et al. (1996). For cryostat sectioning, tissues were embedded in optimal cutting temperature (OTC) compounds (Miles, Inc., Elkart, Ind.), and sections stained for X-Gal were counterstained with haematoxylin and eosin, and mounted.

Targeted Mutagenesis of the Afp Gene

The Afp gene was disrupted by gene targeting in embryonic stem (ES) cells. The lacZ reporter was introduced in Afp gene by homologous recombination and placed under the control of the AFP promoter-enhancer region. The resulting allele is deleted for most of the sequence of exon1, for exon2 and 3 and homologous insertion was detected by Southern analysis. To test the functionality of the reporter one may take advantage of the observation that AFP is expressed in embryoid bodies (Abe et al., 1996). Reporter gene activity is highly turn on in some cells of these bodies.

ES cells Afp lacZ1/+ were injected into C57BL/6J blastocysts. Chimeric animals were obtained and mated with outbred CD1 or inbred 129/CGR to test for germ line transmission. Phenotypically normal heterozygous mice afp lacZ1/+ were generated and detected by Southern blot.

The strain IBMM150 has been submitted to a deposit according to the Budapest Treaty under the deposit number LMGP-19171 (filed on Nov. 29, 1999) at the Belgian Coordinate of Micro-organisms BCCM-LMGP, Laboratorium voor Microbiologie-Bacteriënverzameling, Universiteit Gent, K. L. Ledeganckstraat 35, B-9000 GENT-BELGIUM.

### REFERENCES

Backman, K. & Boyer, H. W., Gene 26, pp. 197-203 (1983)

Bernard P., BioTechniques 21, pp. 320-323 (1996)

Bernard P. et al., Gene 148, pp. 71-74 (1994)

Bravo, A. et al., Mol. Gen. Genet. 215, pp. 146-151 (1988)

Couturier, M. et al., Trends in Microbiology 6, pp. 269-275 (1998)

Gabant, P. et al., BioTechniques 23, pp. 938-941 (1997)

Gabant, P. et al., Gene 207, pp. 87-92 (1998)

Gabant, P. et al., BioTechniques 28(4), pp. 784-788 (2000)

Gossen J. A. et al., Nucleic Acids Res. 20, p. 3254 (1992)

Guilfoyle R. A. & Smith L. M., Nucleic Acids Res. 22, pp. 100-107 (1994)

Guzman L. M. et al., J. Bact. 177, pp. 4121-4130 (1995)

Herrero M. et al., J. Bact. 172, pp. 6557-6567 (1990)

Jensen, R. B. & Gerdes, K., Mol. Microbiol. 17, pp. 205-210 (1995)

Kuhn I. et al., Gene 42, pp. 253-263 (1986)

Meynell, E. & Datta, N., Gen. Res. 7, p. 141 (1966)

Ruiz-Echevarria M. J. et al., J. Mol. Biol. 7, pp. 568-577 (1995)

Simons R. W. et al., Gene 53, pp. 85-96 (1987)

Trudel P. et al., BioTechniques 20, pp. 684-693 (1996)

Tsuchimoto, S. et al., J. Bact. 170, pp. 1461-1466 (1988)

Van Reeth, T., BioTechniques 25, pp. 898-904 (1998)

Vernet T. et al., Gene 34, pp. 87-93 (1985)

Yanish-Perron, C. et al. , Gene 33, pp. 103-109 (1985)

1. Nucleic acid construct (1) to be incorporated in a double selection vector (2) able to transform a cell (3) of a specific organism, wherein
said construct (1) contains two different genes (10 and 11), each gene encoding a different toxic molecule (4 and 5) to a prokaryote cell, said genes (10 and ii) being disposed upstream and downstream a cassette sequence (8), or downstream and upstream site(s) for the insertion of a cassette sequence (8), and
said nucleic acid construct comprises specific sequence portions (12, 12′) allowing inactivation of said genes (10 and 11).

2. The nucleic acid construct according to claim 1, wherein the genes inactivation is obtained through an insertion of a foreigner sequence, such as recombination arms (15, 16), in said genes (10, 11) or through a partial or total deletion of said gene(s) (10, 11).

3. The nucleic acid sequence according to claim 1 or 2, wherein each gene (10 or 11) comprises a prokaryote promoter/operator sequence (9 or 9′) and a sequence encoding a toxic molecule (4 or 5) for said prokaryote cell.

4. The nucleic acid construct according to claim 1 to 3, wherein said genes are disposed in opposite and confluent lecture orientation to each other upstream and downstream the cassette sequence (8).

5. The nucleic acid construct according to any one of the preceding claims, wherein each sequence encoding a toxic molecule (4 or 5) to a prokaryote cell is a nucleotide sequence which encodes a fusion protein active as a poison to the prokaryote cell and made of a coding nucleotide sequence which comprises several unique cloning sites (12) and a nucleotide sequence encoding a protein poison.

6. The nucleic acid construct according to claim 5, wherein the protein poisons are respectively the protein CcdB and the protein Kid.

7. A vector comprising the elements to transform a cell and at least two different genes (10 and 11), each gene encoding a different toxic molecule (4 and 5) to a prokaryote cell.

8. The vector of claim 7 having incorporated the nucleic acid construct according to any one of the preceding claims 1 to 6.

9. The vector according to claim 7 or 8, further comprising, inserted in one of the unique cloning sites of the gene (10), a first recombination arm (15) and inserted in one of the unique cloning sites of the second gene (11) a second recombination arm (16), said recombination arms being able to allow an homologous recombination with corresponding sequences present upstream and downstream a target sequence to be deleted or modified and present in the genome of a cell.

10. A prokaryote cell transformed by the vector according to claim 7 or 8.

11. A prokaryote host cell for the vector according to claim 7 or 8, which
either possesses mutation(s) which confer(s) resistance to the toxic activity of two toxic molecules (6 and 7) encoded by the two different genes (4 and 5) present in said vector,
or possesses a mutation which confers resistance to the toxic activity of a toxic molecule (6) encoded by one of the two different genes (4) present in said vector and possesses one or more genes that encode one or more molecules which is/are anti-poison of one or more toxic molecules (6 and 7) encoded by two different genes (4 and 5) present in said vector.

12. The prokaryotic host cell according to claim 11, which possesses a mutation wherein the argenic 462 is replaced by a cysteine in the amino-acid sequence of the GyrA polypeptide of the gyrase.

13. The cell according to claim 12, having the deposit number LMGP-19171.

14. A method for the modification and/or the replacement of a target genetic sequence into a cell (3) by a cassette sequence (8), comprising the following steps preferably performed by an automate:
incorporating the nucleic acid construct (1) according any of the preceding claims 1 to 5 into a vector (2),
possibly submitting said vector (2) if circular to a cleaving action upon the first specific sequence portions of said nucleic acid construct (preferably to the action of a restriction enzyme upon the unique cloning site (12) of the nucleic acid construct (1) incorporated in said vector), allowing the cleavage or the partial or total deletion of the first gene (10),
incorporating into said vector (2) a first recombination arm (15) that desactivates the toxic activity of the nucleotide sequence present in the first gene (16),
selecting the vector having integrated in the correct orientation a first recombination arm (15) (by transforming a strain (IBMM140) which is sensible to the toxic activity of the first gene (10) but which is resistant to the toxic activity of the second gene (11)),
submitting the recovered vector to a cleaving action upon the second specific sequence portions of said nucleic acid construct (preferably to the action of a restriction enzyme upon the unique cloning site (12′) of the nucleic acid construct (1) incorporated in said vector), allowing the cleavage or the partial or total deletion of the second gene (11),
incorporating into said vector (2) a second recombination arm (16) (or a vector comprising it) that desactivates the toxic activity of the nucleotide sequence present in the second gene (11),
selecting the vector having integrated in the correct orientation the second recombination arm (16) (by transforming a strain that is sensible to the toxic activity of the second gene (11)),
cloning said vector (2) in a procaryote cell, preferably in E. coli, and
transforming a non-human totipotent or a non-human somatic cell (3) in conditions allowing the replacement of a target nucleotide sequence present in the genome of said cell by the cassette sequence (8) of the nucleic acid construct (1) present in said vector (2) by homologous recombination between the arms (15 and 16) and corresponding nucleotide sequences present upstream and downstream the target sequence to be modified or deleted in the genome of said cell (3),
recovering the somatic or totipotent cell (3) wherein the target sequence has been replaced by the cassette sequence (8), and
possibly obtaining a non-human pluricellular organism made of said cell (3) or having incorporated said cell (3) and comprising in its genome the deletion or the modification of the target nucleotide sequence, replaced by the cassette sequence (8) of the nucleic acid construct (1).

15. The method according to claim 14, which further comprises the preliminary steps of:
selecting said target genetic sequence from a genome databases through analysis of said genomic sequence by the identification of exon-intron-structure and comparison with expression genetic databases,
providing the primer sequences suitable for the amplification and cloning of said target genetic sequence,
providing the design of the vector or a nucleic acid construct comprising the nucleotide sequence (8) disposed between suitable recombination arms (15 and 16), and recovering the design of the obtained virtual vector into a target memory database, and
obtaining the suitable means for the preparation of obtained selected nucleic acid construct and vector.

16. A non-human cell or non-human organism obtained by the method according to the claim 14 or 15 and having preferably incorporated into their genome fragments of the vector according to any of the preceding claims 7 or 8, said fragments being preferably two procaryote promoter/operator sequences, preferably from E. Coli, disposed in divergent and opposite lecture orientation.

17. The cell according to the claim 16, being a non-human totipotent cell, preferably a pluripotent embryonic stem (ES) mice cell.

18. A non-human pluricellular organism comprising or made of the cell according to claim 17, preferably a non-human mammal, preferably a mouse.

19. A kit of parts comprising the nucleic acid construct according to the claims 1 to 6, the linear or circular vector according to the claims 7 to 9 and/or means or media for performing the method for the modification or the replacement of a target genetic sequence into a cell by a cassette sequence (8) according to the method of claim 15.

20. Computer program comprising program code means for performing the steps of the method according to the claim 15, when said program is run on a computer.

21. Computer program product comprising the program code means stored on a computer readable medium for performing the steps of the method according to claim 15, when said program is run on a computer.


SEQUENCE LISTING (19 sequences)

SEQ ID NO: 1 - DNA, 25 bp, Artificial Sequence
  construct
acaagtttgtacaaaaaagcaggct

SEQ ID NO: 2 - DNA, 25 bp, Artificial Sequence
  construct
acccagctttcttgtacaaagtggt

SEQ ID NO: 3 - DNA, 29 bp, Artificial Sequence
  kis1 primer
gaggaattctggaggtgaagaatgcatac

SEQ ID NO: 4 - DNA, 29 bp, Artificial Sequence
  kis2 primer
gagaagctttcagatttcctcctgaccag

SEQ ID NO: 5 - DNA, 28 bp, Artificial Sequence
  lambda1 primer
tagagatctgatgcataatgtgcctgtc

SEQ ID NO: 6 - DNA, 27 bp, Artificial Sequence
  lambda2 primer
tagagatctgagcaaaaacaggaaggc

SEQ ID NO: 7 - DNA, 30 bp, Artificial Sequence
  Tn1 primer
taggcggccgcgatgcataatgtgcctgtc

SEQ ID NO: 8 - DNA, 30 bp, Artificial Sequence
  Tn2 primer
taggcggccgccagaagccactggagcacc

SEQ ID NO: 9 - DNA, 390 bp, Artificial Sequence
  fusion poison protein nucleic acid sequence
atgaccatgattacgaattatttaaatcgcgagtcgacggccgcagtggccagcttattggaaagaggggaaatctggcttgtctcgcttgatcctaccgcaggtcatgagcagcagggaacgcggccggtgctgattgtcacaccggcggcctttaatcgcgtgacccgcctgcctgttgttgtgcccgtaaccagcggaggcaattttgcccgcactgccggctttgcggtgtcgttggatggtgttggcatacgtaccacaggtgttgtacgttgcgatcaaccccggacaattgatatgaaagcacggggcggaaaacgactcgaacgggttccggagactatcatgaacgaagttcttggccgcctgtccactattctgacttga

SEQ ID NO: 10 - protein, 129 aa, Artificial Sequence
  fusion poison protein nucleic acid sequence
MTMITNYLNRESTAAVASLLERGEIWLVSLDPTAGHEQQGTRPVLIVTPAAFNRVTRLPVVVPVTSGGNFARTAGFAVSLDGVGIRTTGVVRCDQPRTIDMKARGGKRLERVPETIMNEVLGRLSTILT

SEQ ID NO: 11 - DNA, 36 bp, Artificial Sequence
  MCS34A primer
aattgtttaaacgcccgggcgcggccgcggcgcgcc

SEQ ID NO: 12 - DNA, 36 bp, Artificial Sequence
  MCS34B primer
agctggcgcgccgcggccgcgcccgggcgtttaaac

SEQ ID NO: 13 - DNA, 27 bp, Artificial Sequence
  Plac1 primer
gagagagatctcgcaacgcaattaatg

SEQ ID NO: 14 - DNA, 32 bp, Artificial Sequence
  KIDAX primer
gagacatgtctcgagtcaagtcagaatagtgg

SEQ ID NO: 15 - DNA, 10 bp, Artificial Sequence
  Adapter
tcgaagatct

SEQ ID NO: 16 - DNA, 36 bp, Artificial Sequence
  MCS12A primer
aattatttaaatcgcgagtcgacggccgcagtggcc

SEQ ID NO: 17 - DNA, 36 bp, Artificial Sequence
  MCS12B primer
agctggccactgcggccgtcgactcgcgatttaaat

SEQ ID NO: 18 - DNA, 31 bp, Artificial Sequence
  N-Mer1 primer
agagcggccgcggaagtgacaaagcagaacc

SEQ ID NO: 19 - DNA, 29 bp, Artificial Sequence
  X-exon1 primer
agactcgagggatgagggaagcgggtgtg